(19) United States
(12) Patent Application Publication
(10) Pub. No.: US 2006/0115860 A1
(43) Pub. Date: Jun. 1, 2006

(54) Immunologic assay for detection of autoantibodies to folate binding protein

(76) Inventors: Robert M. Cabrera, Spring, TX (US); Richard Finnell, Houston, TX (US)

Correspondence Address: Benjamin Aaron Adler;ADLER & ASSOCIATES, 8011 Candle Lane, Houston, TX, 77071, US

(21) Appl. No.: 11/288,014

(22) Filed: Nov. 28, 2005

Related U.S. Application Data
(60) Provisional application No. 60/631,130, filed on Nov. 26, 2004.

(51) Int. Cl. G01N 33/53; G01N 33/537; G01N 33/543

(52) U.S. Cl. 435/7.5; 435/7.92

(57) ABSTRACT

The present invention is directed to an assay that detects autoantibodies to folate receptor and can be used in the clinical diagnostic testing of these autoantibodies in humans. Although there are other methods that exist to detect these autoantibodies, the assay described in the present invention has several features that offer advantages over the existing methods. Some of these features include adaptability to high-throughput processing, the use of an immunoglobulin antibody to bind autoantibodies bound to folate receptor or the use of enzyme-labeled folic acid to bind folate binding protein and use of fluorescence or chemiluminescence for detection. This assay thereby avoids the use of radioactivity and can be automated and scaled to process hundreds of samples safely and simultaneously.

### CROSS REFERENCE TO RELATED APPLICATIONS

This non-provisional application claims benefit of provisional application U.S. Ser. No. 60/631,130, filed on Nov. 26, 2004, now abandoned.

### BACKGROUND OF THE INVENTION

1. Field of the Invention

The present invention relates generally to the field of immunology. More specifically, the present invention uses Enzyme Linked Immunosorbent Assay (ELISA) technology to detect the presence of autoantibodies to folate binding proteins by non-radioactive means.

2. Description of the Related Art

Neural tube defects, which include spina bifida, anencephaly, craniorachischisis and encephalocele, occur in approximately 1 per 1000 births in the United States. Additionally, women who have one fetus with this complication are at increased risk in subsequent pregnancies (1). There are multiple causes of neural tube defects including drugs, especially antifolate (2) and antiepileptic (3) agents, chromosomal abnormalities (Seller M. J., 1995), and environmental (5) and genetic factors (6). Although periconceptional folic acid supplementation reduces the occurrence and recurrence of neural tube defects by approximately 70 percent (7-8), most women who are pregnant with a fetus with this complication do not have clinical folate deficiency (9). Though some polymorphisms for folate-pathway enzymes (10) have been identified, they cannot account for the 70 percent decrease in the incidence of this birth defect with folate supplementation.

Studies in animals have suggested the importance of folate receptors in embryogenesis (11). Inactivation of both alleles encoding the mouse homologue of human folate receptor a gene was uniformly fatal in embryos with neural-tube defects (12-13). Folinic acid given to pregnant dams resulted in normal development in 80 percent of the embryos that lacked folate receptor a gene in both alleles (13). However, no specific polymorphism or mutations of the human folate receptor gene have been identified that might explain the reduction in the incidence of neural tube defects with folic acid supplementation (14).

Administration of an antiserum to folate receptors (15) to pregnant rats resulted in the resorption of or multiple developmental abnormalities in embryos (16). This observation led to the speculation that an autoantibody against folate receptors in women could induce similar embryonic and fetal abnormalities. The speculation was confirmed when autoantibodies against folate receptors were detected in serum from women who had a pregnancy complicated by a neural-tube defect (17). In this study, 40 μl of treated serum was incubated overnight with a [³H] folic acid-folate receptor complex. Additionally, staphylococcal protein A membranes was used to precipitate a [³H] folic acid-folate receptor complex and the radioactivity of the sample was then detected. A modification of this method involved incubating 30-60 μl of treated sample overnight with folate receptor. Radioactive [³H] folic acid was then added to the solution followed by incubation at room temperature for 30 minutes. The unbound folic acid was removed and the radioactivity remaining in the supernatant fraction measured. However, both these methods offered limited sample processing, used radioactive folate and thereby posed environmental and safety risks.

The prior art is deficient in a non-radioactive, automated method that detects autoantibodies to the folate receptor and which could process hundreds of samples safely and simultaneously. The present invention fulfills this long-standing need and desire in the prior art.

### SUMMARY OF THE INVENTION

The present invention is directed to a process that detects autoantibodies to the folate receptor. The features of this process that make it advantageous over the existing process used for detection of folate receptor antibodies include the adaptability to high-throughput processing, the use of an enzyme- or fluorescently-labeled ligand to determine the presence or absence of autoantibodies bound to folate receptor and the use of fluorescence or chemiluminescence for detection.

In one embodiment of the present invention, there is a high-throughput assay for detecting autoantibodies to folate receptor in serum of an individual. In this assay, folate binding protein solution is deposited onto plates, where the surfaces of the plates are modified to form covalent bonds with the folate binding proteins. The serum is then applied to the protein deposited plates. Further, a labeled biomolecule is added to the serum-applied plates. Finally, substrate for the labeled biomolecule is added. This substrate detects interactions between the labeled biomolecule, the autoantibodies and the folate binding proteins. All of this enables detection of the autoantibodies to the folate binding proteins in the serum.

In another embodiment of the present invention, there is a diagnostic kit to detect autoantibodies to the folate receptor in the serum of an individual. The kit comprises: (a) surface-modified or surface-coated plates, (b) folate binding protein, (c) labeled biomolecule, and (d) substrate for the labeled biomolecule.

### BRIEF DESCRIPTION OF THE DRAWINGS

So that the matter in which the above-recited features, advantages and objects of the invention, as well as others which will become clear, are attained and can be understood in detail, more particular descriptions of the invention are briefly summarized. The above may be better understood by reference to certain embodiments thereof which are illustrated in the appended drawings. These drawings form a part of the specification. It is to be noted; however, that the appended drawings illustrate preferred embodiments of the invention and therefore are not to be considered limiting in their scope.

FIG. 1 shows a glass slide with a hydrophobic coating (Teflon) to produce a 96-well format.

FIGS. 2A-C show changes to the hydrophobic “footprint”. FIG. 2A shows a 16-well format (microscope slide). FIG. 2B shows a 384-well format. FIG. 2C shows a 1536-well format.

FIG. 3 shows testing of an ELISA Based Assay with folate-binding proteins from human, mouse and cow. Samples were applied as represented in the figure as negative (1), medium titer positive (2), and intermediate titers (3, 4). Human folate-binding protein was printed in column 1, mouse folate-binding proteins in column 2 and cow folate-binding proteins in column 3. Column 4 was a negative control in which print buffer without folate-binding proteins was printed. Arrays were processed and imaged under UV light.

FIGS. 4A-D show results from control and experimental sera testing. Values were calculated from 8-bit images. All intensities are local background subtracted. FIGS. 4A and 4B demonstrate the reproducibility and sensitivity of detected intensities by analysis of the medium titer serial dilution. FIG. 4C shows detected intensities across duplicate wells for each of the 40 samples. FIG. 4D displays the averaged results for each sample after it was scaled to the serial dilution of the medium titer positive control. The negative (Neg) is a buffer control in which no serum was present.

FIG. 5 shows the results from a variant of the assay. Proteins were immobilized to a microscope slide. Interactions were detected via folic acid labeled with horseradish peroxidase. The peroxidase substrate used for detection was cyanine 3 tyramide. Images were collected using a laser scanner and intensities were determined from the generated 16-bit images. Tetanus toxoid is shown as a negative control in this figure and thus, exhibited no folic acid binding. Decreasing the available binding sites for enzyme labeled folic acid by the addition of a competitive binder, e.g., percent of unlabeled folic acid, reduced the detected signal. The resultant dilution curves show R² values of 0.94 for human (Homo) and 0.96 for bovine (Bevo) folate receptors.

### DETAILED DESCRIPTION OF THE INVENTION

Neural-tube defects are caused by multiple factors (2-6). Studies showing a reduction in the incidence of neural tube defects of approximately 70 percent with periconceptional folic acid supplementation (7-8) provide evidence that supplementary folate circumvents either an impaired intracellular folate-dependent enzyme pathway or an inhibitor of the cellular uptake of folate. However, the genetic variants of folate pathway enzymes or of folate receptors identified in women who have pregnancies complicated by neural tube defect do not account for the 70 percent reduction in neural tube defects associated with folate supplementation (18).

Additionally, one study identified autoantibodies against folate receptors in serum from women who had pregnancy complicated by a neural-tube defect (17). However in this study, the treated serum was incubated with radioactive [³H] folic acid-folate receptor complex, which was then precipitated with staphylococcal protein A and the radioactivity of the sample detected. Alternatively, instead of incubating serum with radioactive [³H] folic acid-folate receptor complex, one could incubate the serum with folate receptor followed by addition and incubation with [³H] folic acid. The radioactivity remaining in the supernatant could be measured after removing the unbound radioactive folic acid. Therefore, these methods in addition to offering limited sample processing pose environmental and safety risks due to use of radioactivity.

The present invention applied protein array technology to detect the presence of autoantibodies to folate binding proteins. This high-throughput format for testing the serum samples described in the present invention was adapted from another method (19). Additionally, the method described in the present invention requires printing folate-binding proteins (folbp) directly onto 96-well array, which enables detection and determination of the relative quantity of folate-binding proteins autoantibodies in human sera in a reproducible and high-throughput manner. Furthermore, the method described in the present invention uses either labeled immunoglobulin antibody that binds autoantibodies bound to folate binding protein or labeled folic acid that binds the folate binding protein in one of the steps leading to detection of the autoantibodies. This high-throughput assay can be used in the clinical diagnostic testing of folate-binding proteins autoantibody in humans.

Using an ELISA based assay, the present invention demonstrated that folate-binding proteins from human, mouse and cow could be utilized as probes for folate-binding proteins autoantibodies. Additionally, by using sera samples that had been previously tested by radiological method, the method of the present invention categorized these sera based on the autoantibody titer. Based on the R² values, this method also demonstrated high reproducibility and sensitivity for detecting antibodies down to the 1:32 dilution.

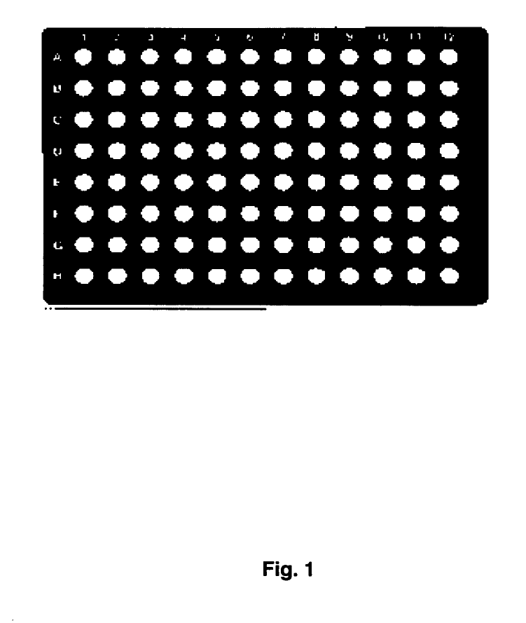

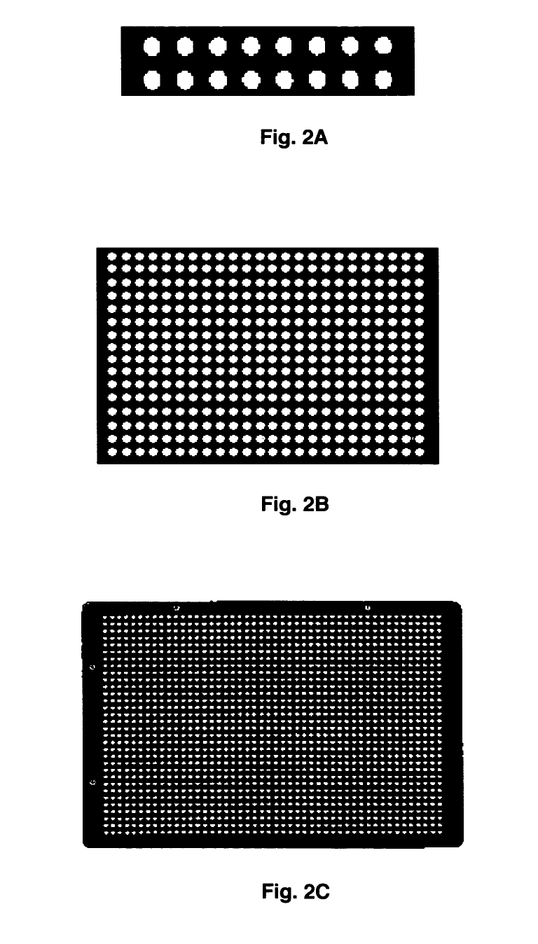

Therefore, this method has several advantages over the previously used method (17). First, this method can be automated and scaled to process hundreds of samples simultaneously as opposed to limited sample processing by the previously used method. Second, since this method uses fluorescence to detect autoantibodies to folate receptors in the serum samples, it is much safer than the previously described methods, which use radioactive folate. Third, this method requires only 1 μL of serum per assay and therefore 10 μL provides enough working solution for 10 assays. Fourth, this method allows processing of samples in a high-throughput multi-well format (FIGS. 1, 2A-2C). Fifth, by not requiring overnight processing of the samples, this method is much faster and can provide results in less than 4 hours. Sixth, this method differs from the previously used method since it uses immunoglobulin antibody to detect the autoantibodies, where the immunoglobulin antibody as well as it's labeling can be varied.

The present invention is directed to a high-throughput assay for detecting autoantibodies to folate receptor in serum of an individual, comprising: depositing folate binding protein solution onto plates, where the surface of the plates are modified to form covalent bonds with the folate binding proteins, applying the serum onto the protein deposited plates, adding labeled biomolecule to the serum-applied plates and adding substrate for the labeled biomolecule, where the substrate detects interactions between the labeled biomolecule, the autoantibodies and the folate binding proteins, thereby detecting the autoantibodies to the folate receptor in the serum. This assay further comprises: processing the serum to remove endogenous soluble folate binding proteins and endogenous folate. Examples of the labeled biomolecule is not limited to but includes a labeled immunoglobulin antibody that binds autoantibodies bound to the folate binding proteins deposited on the plates or is an enzyme- or fluorescently-labeled folic acid that binds the folate binidng proteins deposited on the plates.

Furthermore, the labeled immunoglobulin antibody binds autoantibodies bound to the folate binding proteins that are immobilized on the plates modified with 1% solution of (3-glycidoxypropyl)trimethoxysilane in toluene. Examples of the labeled immunoglobulin antibody are not limited to, but include, a labeled IgG, a labeled IgM or a labeled IgA immunoglobulin antibody. Additionally, the labeled IgG immunoglobulin antibody is a labeled IgG1, labeled IgG2, labeled IgG3, or labeled IgG4 immunoglobulin antibody. Further, the immunoglobulin antibody is labeled with fluorescent dye, Digoxigenin, anti-Digoxigenin, alkaline phosphatase, peroxidase, avidin, streptavidin, or biotin. Additionally, the alkaline phosphatase labeled immunoglobulin antibody has anti-IgG immunoglobulin activity. Furthermore, a substrate for the labeled immunoglobulin antibody includes, but is not limited to, a chemiluminescent or a fluorescent phosphatase or a peroxidase substrate or a fluorescent dye labeled with Digoxigenin, anti-Digoxigenin, biotin, avidin or streptavidin. Specifically, the fluorescent phosphatase substrate is ELF97 phosphatase substrate.

Furthermore, the labeled biomolecule comprising a labeled folic acid is folic acid labeled with fluorescent dye, alkaline phosphatase or a horseradish peroxidase. A substrate for the enzyme labeled folic acid includes, but is not limited to, a fluorescent phosphatase or a chemiluminescent horseradish peroxidase substrate. Generally, the folate binding proteins bound by the enzyme-labeled folic acid are labeled prior to being deposited onto plates. Specifically, the folate binding proteins labeled with biotin are deposited onto streptavidin-coated plates. Additionally, the folate binding protein is isolated from vertebrate species selected from the group consisting of human, mouse cow, pig and monkey. Further, the high-throughput assay is a multi-format assay. The multi-well format assay comprises standard microtiter high throughput dimensions or standard microtiter ultrahigh throughput dimensions. Generally for an assay of this kind, the plate used could be microarray, microtiter or any other structure suitable for binding folate binding protein as would be well-known in the art.

The present invention is also directed to a diagnostic kit to detect autoantibodies to the folate receptor in serum from an individual. This kit comprises: (a) surface-modified or surface-coated plates, (b) folate binding protein, (c) labeled biomolecule and (d) substrate for the labeled biomolecule. Generally, the labeled biomolecule is a labeled immunoglobulin antibody that binds autoantibodies bound to the folate binding proteins deposited on the surface-modified plates or is an enzyme or fluorescently-labeled folic acid that binds the folate binding proteins deposited on the surface-coated plates. Additionally, the surface-modified plates in the kit comprising labeled immunoglobulin antibody are surface-modified microarray or surface-modified microtiter plates. The surfaces of such plates are modified using a 1% solution of (3-glycidoxypropyl)trimethoxysilane in toluene.

The labeled IgG immunoglobulin antibody is a labeled IgG1, labeled IgG2, labeled IgG3, or labeled IgG4 immunoglobulin antibody. Furthermore, the immunoglobulin antibody is labeled with fluorescent dye, Digoxigenin, anti-Digoxigenin, alkaline phosphatase, peroxidase, avidin, streptavidin or biotin. Additionally, the alkaline phosphatase labeled immunoglobulin antibody has anti-IgG immunoglobulin activity. Generally, a substrate for the labeled immunoglobulin antibody includes but is not limited to a chemiluminescent or a fluorescent phosphatase or a peroxidase substrate or a fluorescent dye labeled with Digoxigenin, anti-Digoxigenin, biotin, avidin or streptavidin. Specifically, the fluorescent phosphatase substrate is ELF97 phosphatase substrate.

As discussed supra, the kit comprises a labeled folic acid as a labeled biomolecule. Generally, the folic acid is labeled with an alkaline phosphatase or a horseradish peroxidase. Additionally, a substrate for the labeled folic acid includes but is not limited to a fluorescent phosphatase or a chemiluminiscent horseradish peroxidase. The folate binding protein in such a kit is labeled with biotin prior to being deposited on streptavidin coated plates. The streptavidin coated plates in the kit may be microtiter plates. Additionally, the folate binding proteins in both the kits are isolated from vertebrate species. These vertebrate species are selected from a group consisting of a human, mouse, cow, pig, and monkey.

As used herein, the term, “a” or “an” may mean one or more. As used herein in the claim(s), when used in conjunction with the word “comprising”, the words “a” or “an” may mean one or more than one.

As used herein, the term, “labeled biomolecule” refers to a molecule labeled with an enzyme or a fluorescent dye or a protein that binds either to the autoantibodies bound to the folate binding proteins or to the folate binding proteins and detects autoantibodies in the serum on addition of appropriate substrate. The present invention demonstrates the utility of labeled immunoglobulin antibody and labeled folic acid as examples of a labeled biomolecule that could be used in the method or kit described herein.

As used herein, the term “substrate” refers to any compound that is added to the labeled biomolecule and detects the interaction between the labeled biomolecule, the autoantibodies in the serum and the folate binding protein. Such a substrate may be a fluorescently or chemiluminscently labeled substrate for a particular enzyme that the biomolecule is labeled with or is a fluorescent dye that is labeled with proteins that can form complexes with the protein that the biomolecule is labeled with.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Testing of an ELISA-Based Assay with Folate Binding Protein (Folbp) from Human, Mouse and Cow

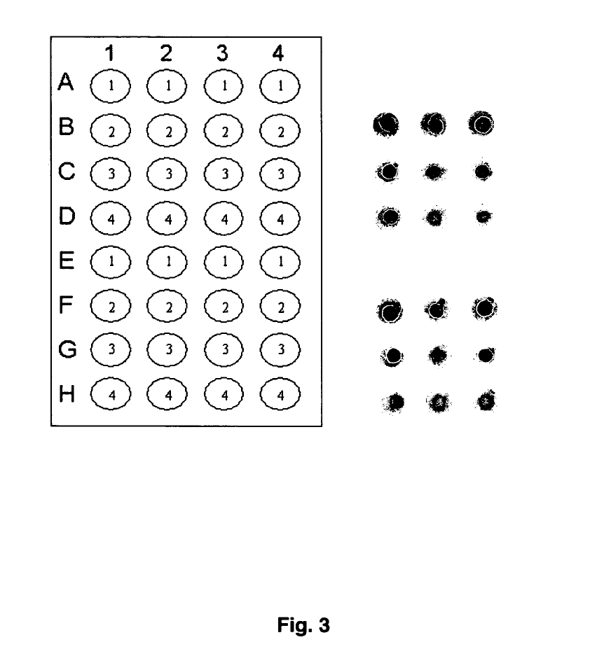

Folbp were obtained from human, mouse and cow. The human folbp was isolated from human placenta as described previously (20) and was made available for testing by Dr. Sheldon P. Rothenberg. Both the mouse folate-binding proteins (Orthoclinical Diagnostics, Raritan, N.J.) and cow folate-binding proteins (Sigma Aldrich, St. Louis, Mich.) were obtained commercially. Sera for preliminary tests were graciously donated and had been previously tested in a published radiological method to detect autoantibodies (17). Results of this preliminary test indicated that the ELISA based assay was functional and demonstrated that all proteins tested could be utilized as probes for folate-binding proteins autoantibodies (FIG. 3).

### EXAMPLE 2

Analysis of Human Serum for Folbp Autoantibodies

Serum samples were obtained from women during mid-gestational pregnancy and the samples were tested to identify presence, absence and relative abundance of folate-binding proteins autoantibodies in them.

For the fabrication of microarray plates, glass 96-well microarray plates were purchased commercially (Precisions Lab Products, Middleton, Wis.) for array fabrication. Briefly, using glass wash chambers the microarray plates were rinsed thrice with Milli-Q Ultrapure Water (Millipore Bilerica, MA). The slides were then rinsed thrice in 100% ethanol, followed by rinsing twice in toluene. A 1% solution of (3-glycidoxypropyl)trimethoxysilane in toluene was prepared fresh for surface modification to the silica microarray plate surface. Attachment of the monolayer was allowed to proceed overnight (14-16 hours). The slides were then rinsed twice in toluene, followed by rinsing twice in 100% ethanol. Slides were then dried under filtered argon. It has been previously shown that this method of fabrication produces monolayers of epoxysilane films (21). The slides were then utilized for coupling proteins to the surface immediately after drying.

For the printing of folate binding protein, bovine folate binding protein (B-folbp) was purchased commercially (Sigma Aldrich) for binding to epoxysilane surface. The B-folbp was suspended in 1× phosphate buffered saline with 5 mM sodium azide to produce 1 mg/ml stock solution. For printing of the B-folbp, the stock solution was diluted in 50 mM NaHCO₃ (pH 9.6) at 5 μg/ml. The probe was then mechanically deposited onto the array in 0.5 μL volumes under ambient conditions inside a polycarbonate cabinet. After the probe had dried, the slides were pre-imaged using light microscopy in order to examine spot morphology. For quality control purposes, irregular or missing spots were flagged and these wells were not used for sample processing. The slides were then stored under desiccant at 4° C.

Serum samples were prepared by adding 490 μL of 100 mM citric acid buffer (pH 3.0) to 10 μL aliquot of the sample. This pH was used to allow dissociation of antibodies. Additionally, it has also been shown that folate receptor dissociates from endogenous folate at this pH (20). The serum-buffered solution was then fractioned using Microcon-100 filters (Millipore) and only those proteins that are above 100 KD were retained. Since the average IgG immunoglobulin was 150 KD, the 100 KD cutoff allowed antibody retention while removing endogenous folate and soluble folbp. The samples were washed once with 500 μL of 5 mM citric acid buffer (pH 3.0) followed by a wash with 500 μL of 100 mM NaHCO₃ (pH 8.3). The fractioned serum sample was then collected in 20 μL of 100 mM NaHCO₃ buffer (pH 8.3). All spin times used in this procedure were according to manufacturer's recommendations (washes at 12 minutes×14,000 G, collection at 3 minutes×1000 G). The 20 μL fraction of serum was brought up to 250 μL by adding 1.25 μL of 200 mM Phenylmethylsulfonyl Fluoride in DMSO (1 mM Final) and 228.75 μL SuperBlock Blocking Buffer (Pierce-Rockford, Ill.). Since a total of 25 μL of this solution was used per assay, 10 μL of whole serum yielded enough working solution for 10 assays.

Before application of the working solution, non-bound folbp was removed from wells by two washes with 1×TNT-Glycine buffer (100 mM Tris-HCl pH 7.6, 150 mM NaCl, 0.05% Tween-20, 20 mM Glycine). Unless indicated otherwise, all solution volumes were 25 μL per well. The surface was then blocked by the addition of 1×TNT-glycine buffer for 1 hour. After blocking, the wells were washed with 1×TNT thrice, followed by addition of the serum working solution to the slides. The slides and the serum working solution were then incubated in a polycarbonate cabinet for two hours under ambient conditions. Following the incubation period, the wells were washed five times with 1× TNT. A secondary conjugate labeled with alkaline phosphatase and specific to the detection of human IgG immunoglobulins was diluted in 1× TNT and then applied (25 μL per well) according to the manufacturer's ELISA recommendations (SigmaAldrich). The slides and the secondary antibody solution were then incubated in a polycarbonate cabinet for one hour under ambient conditions. Following the incubation period, the wells were washed seven times with 1× TNT.

Detection of the interactions between folate binding protein, autoantibodies and the alkaline phosphatase IgG secondary conjugate was assayed using the ELF 97 phosphatase substrate (Molecular Probes-Eugene, Oreg.). The ELF 97 substrate (component D) was used with accompanying in-situ hybridization buffers. The substrate was diluted ten fold into Buffer C and filtered through a syringe filter (0.2 μm) to remove precipitates. Immediately before use, Components E and F (1:500 dilution) were added to the filtered solution and the substrate was then applied to the slides at 20 μL per well. The slides with applied substrate were then incubated in a polycarbonate cabinet for 30 minutes under ambient conditions. Following this incubation period, the slides were rinsed once with 1× TNT followed by a Milli-Q Ultrapure water rinse. Slides were then imaged using a UV photography workstation (Kodak).

For the analysis of data, probe intensities were determined using Scion Image (Frederick, Md.). All features intensities were calculated as foreground minus local background. A serial dilution of a control sample was used on all slides as a reference. The control consisted of a medium titer antibody positive serum and allowed determination of assay sensitivity and relative concentration of antibodies in experimental samples. All extracted data was analyzed as raw data and was then transformed to a relative “fold-dilution” concentration. Results above 1:2 dilutions were categorized as positive, between 1:2 and 1:8 dilutions were categorized as intermediate and below an 8-fold dilution were categorized as negative. The lower threshold of relative detection was between the 1:32 to 1:64 dilutions. Extrapolation of data values above 1 were calculated according to relative exponential regression line; therefore, in all tested samples the raw data for values were also included in order to negate reference categorization bias and error based on extrapolation. All analyses and data presentation were performed using Microsoft Office Suite 2000 (Microsoft Corp-Redmond, Wash.).

### EXAMPLE 3

Preliminary Analysis of Human Serum for Folbp Autoantibodies

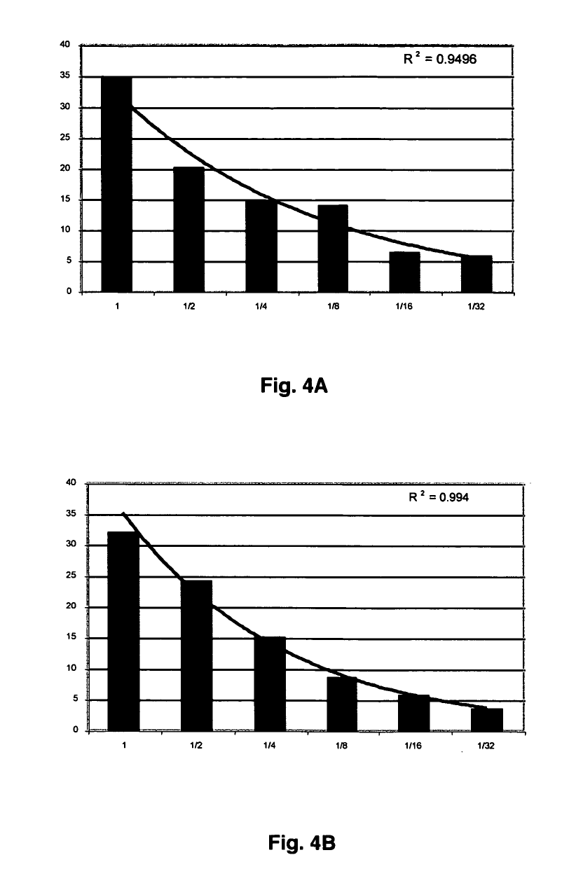

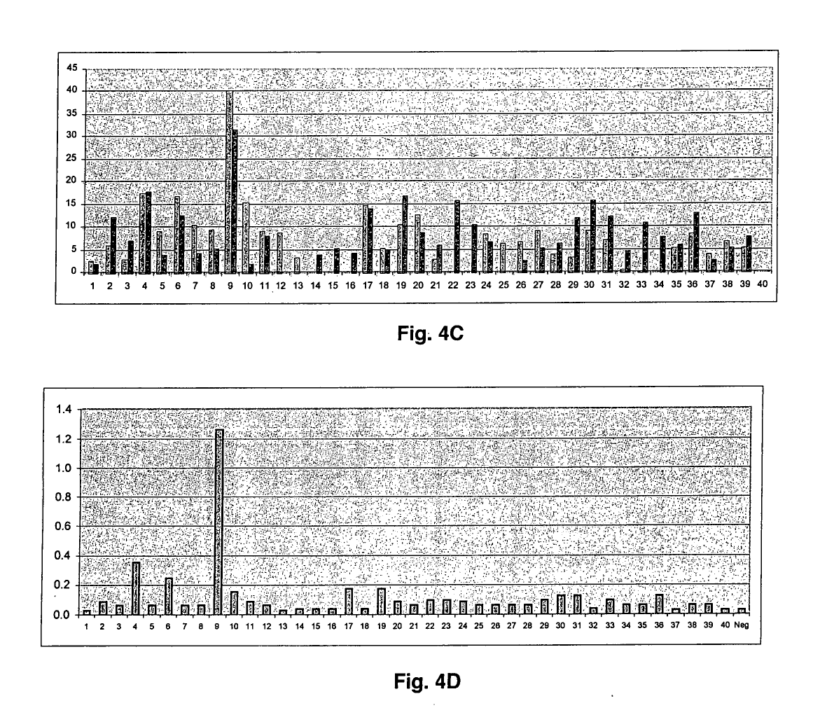

In order to test the assay under clinical conditions, 40 sera samples were obtained for testing as mentioned earlier. Arrays samples were obtained from women in mid-pregnancy from 16 to 45 years of age. All samples were obtained from the California Birth Defects Monitoring Program with informed consent. Arrays and samples were prepared as described above and tested in duplicate. This analysis allowed estimation of the assays sensitivity and reproducibility through the use of standard dilutions of the medium titer positive control sample (FIGS. 4A-4D). Results indicated that 1 serum sample tested positive (#9) for autoantibodies and 5 sera tested intermediate. This latter group was characterized by 1 high-intermediate titer (#4) (between 1:2 to 1:4) and 4 low-intermediate titers (#6, 10, 17, 19) (between 1:4 to 1:8) when compared to the standard dilution of the medium titer control serum diluted from 1 to 1:64. As indicated by the R² values of the controls, the assay demonstrated high reproducibility and sensitivity for detecting antibodies down to the 1:32 dilution (FIGS. 4A-4B).

### EXAMPLE 4

Alternate Assay to Detect Folbp Autoantibodies

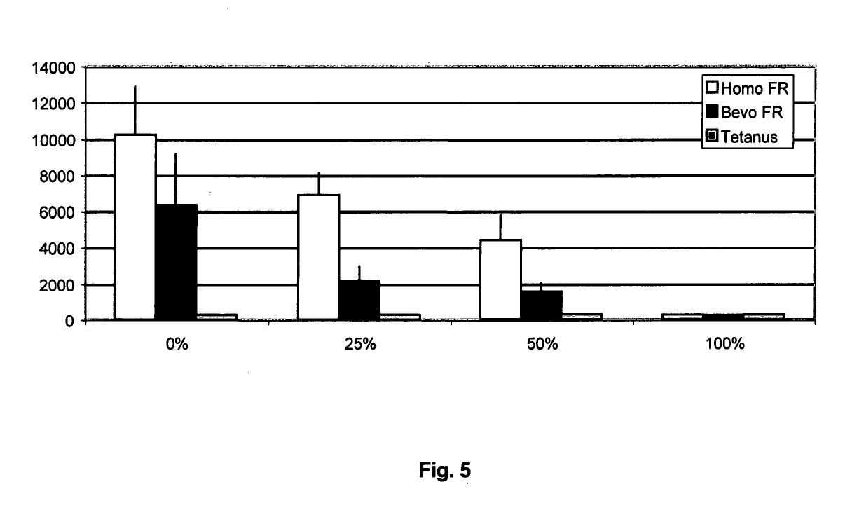

In order to test a variation of the described assay, arrays and sera were prepared as described above. Interactions with the immobilized receptors were detected via folic acid labeled with horseradish peroxidase (HRP). The peroxidase substrate used for detection was Cyanine 3 tyramide (PerkinElmer Life and Analytical Sciences, Boston, Mass.). Images were collected using a laser-scanner (Genomic Solutions, Ann Arbor, Mich.) and intensities were determined from the generated 16-bit images. Results allowed an estimation of the reproducibility of the assay and indicated that both bovine and human folate receptors bound folic acid with high specificity (FIG. 5). Decreasing the available binding sites for enzyme labeled folic acid by addition of a competitive binder reduced the resultant signal. Representative examples of such competitive binders include antibodies against folate receptor, sera with antibodies directed against folate receptor, and unlabeled folates. Unlabeled folic acid was spiked into antibody-deplete sera obtained commercially (Sigma, St. Louis, Mo.) in order to generate a standard curve (FIG. 5). The curve represented the amount of folic acid-HRP blocked by unlabeled folic acid in antibody-deplete sera. The ability of antibodies in experimental sera to block folic acid binding is determined relative to this standard curve.

The following references were cited herein:
1. Cragan, J. D. et al. (1995) MMWR CDC Surveill Summ 44(4):1-13.
2. Hernandez-Diaz et al. (2000), N Engl J Med 343, 1608-1614.
3. Dansky, L. V. et al. (1992) Neurology 42 (Suppl 5), 32-42.
4. Seller, M. J. (1995) Clin Dysmorphol 4, 202-207.
5. Finnell, R. H. et al. (2000) Ann N Y Acad Sci. 919, 261-277.
6. De Marco, P. et al. (2000) Am J Med Genet 95, 216-223.
7. Czeizel A. E. and Dudás I. (1992) N Engl J Med 327, 1832-1835.
8. MRC Vitamin Study Research Group. (1991) Lancet 338, 131-137.
9. Giles, C. (1966) J Clin Path 19, 1-11.
10. Christensen, B. et al. (1999) Am J Med Genet 84, 151-157.
11. Antony, A. C. (1996) Ann Rev Nutr 16, 501-521.
12. Piedrahita, J. A. (1999) Nat Genet 23, 228-232.
13. Finnell, R. H. et al., (2002) Folate transport abnormalities and congenital defects. In: Milstien et al., eds. Chemistry and biology of pteridines and folates. Boston: Kluwer Academic, 637-642.
14. Barber, R. C. et al. (1998) Am J Med Genet 76, 310-317. (Erratum, Am J Med Genet 1998, 79, 231.J).
15. da Costa, M. and Rothenberg, S. P. (1996) Biochim Biophys Acta 1292, 23-30.
16. da Costa, M. et al. (2003) Birth Defects Res Part A Clin Mol Teratol 67, 837-847.
17. Rothenberg, S. P. et al. (2004) N Engl J Med 350 (2), 134-142.
18. Antony, A. C. and Hansen, D. K. (2000) Teratology 62, 42-50.
19. Mendoza, L. G. et al. (1999) Biotechniques 27, 778-788.
20. Sadasivan et al. (1987) Biochim Biophys Acta 925, 36-47.
21. TsukrukV. et al. (1999) ACS 15, 3029-3032.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A high-throughput assay for detecting autoantibodies to folate receptor in serum of an individual, comprising:
depositing folate binding proteins onto plates, wherein the surfaces of the plates are modified to form covalent bonds with the folate binding proteins;
applying said serum onto said protein deposited plates;
adding a labeled biomolecule to the serum-applied plates;
adding a substrate for said labeled biomolecule, wherein said substrate detects interactions between said labeled biomolecule, said autoantibodies and said folate binding proteins, thereby detecting said autoantibodies to said folate receptor in said serum.

2. The assay of claim 1, further comprising:
processing said serum to remove endogenous soluble folate binding protein and endogenous folate.

3. The assay of claim 1, wherein said labeled biomolecule is a labeled immunoglobulin antibody that binds autoantibodies bound to the folate binding proteins deposited on the plates or is an enzyme or fluorescently labeled folic acid that binds the folate binding proteins deposited on the plates.

4. The assay of claim 3, wherein the labeled immunoglobulin antibody binds autoantibodies bound to the folate binding proteins that are immobilized on the plates modified with 1% solution of (3-glycidoxypropyl)trimethoxysilane in toluene.

5. The assay of claim 3, wherein the labeled immunoglobulin antibody is a labeled IgG, a labeled IgM or a labeled IgA immunoglobulin antibody.

6. The assay of claim 5, wherein said labeled IgG immunoglobulin antibody is a labeled IgG1, labeled IgG2, labeled IgG3, or labeled IgG4 immunoglobulin antibody.

7. The assay of claim 3, wherein said immunoglobulin antibody is labeled with a fluorescent dye, Digoxigenin (DIG), anti-Digoxigenin, alkaline phosphatase, peroxidase, avidin, streptavidin, or biotin.

8. The assay of claim 7, wherein said alkaline phosphatase labeled immunoglobulin antibody has anti-IgG immunoglobulin activity.

9. The assay of claim 3, wherein a substrate for the labeled immunoglobulin antibody is a chemiluminescent or a fluorescent phosphatase or a peroxidase substrate or a fluorescent dye labeled with Digoxigenin (DIG), anti-Digoxigenin, biotin, avidin or streptavidin.

10. The assay of claim 9, wherein said fluorescent phosphatase substrate is ELF 97 phosphatase substrate.

11. The assay of claim 3, wherein the folic acid is labeled with a fluorescent dye, an alkaline phosphatase, or a horseradish peroxidase.

12. The assay of claim 11, wherein a substrate for the enzyme labeled folic acid is a fluorescent phosphatase or a chemiluminescent horseradish peroxidase substrate.

13. The assay of claim 3, wherein the folate binding proteins bound by the labeled folic acid are labeled prior to being deposited onto plates.

14. The assay of claim 13, wherein the folate binding proteins are labeled with biotin and deposited onto streptavidin-coated plates.

15. The assay of claim 1, wherein said folate binding protein is isolated from vertebrate species.

16. The assay of claim 15, wherein said folate binding protein is isolated from a vertebrate species selected from the group consisting of human, mouse, cow, pig, and monkey.

17. The assay of claim 1, wherein said high-throughput assay is a multi-format assay.

18. The assay of claim 17, wherein said multi-format assay comprises standard microtiter high throughput dimensions or standard microtiter ultrahigh throughput dimensions.

19. The assay of claim 18, wherein said multi-format assay is performed using microarray or microtiter plates.

20. A diagnostic kit to detect autoantibodies to folate receptor in serum of an individual, comprising:
(a) surface-modified or surface-coated plates;
(b) folate binding protein;
(c) labeled biomolecule; and
(d) substrate for said labeled biomolecule.

21. The kit of claim 20, wherein the labeled biomolecule is a labeled immunoglobulin antibody that binds autoantibodies bound to the folate binding proteins deposited on the surface-modified plates or is an enzyme or fluorescently labeled folic acid that binds the folate binding proteins deposited on the surface-coated plates.

22. The kit of claim 21, wherein the surface-modified plates in the kit are surface-modified microarrays or surface-modified microtiter plates.

23. The kit of claim 22, wherein the surfaces of the plates are modified with 1% solution of (3-glycidoxypropyl)trimethoxysilane in toluene.

24. The kit of claim 21, wherein said labeled immunoglobulin antibody is a labeled IgG, a labeled IgA or a labeled IgM immunoglobulin antibody.

25. The kit of claim 24, wherein said labeled IgG antibody is a labeled IgG1, IgG2, IgG3, or IgG4 immunoglobulin antibody.

26. The kit of claim 21, wherein said immunoglobulin antibody is labeled with a fluorescent dye, Digoxigenin (DIG), anti-Digoxigenin, alkaline phosphatase, peroxidase, streptavidin, avidin or biotin.

27. The kit of claim 26, wherein said alkaline phosphatase labeled immunoglobulin antibody has anti-IgG immunoglobulin activity.

28. The kit of claim 21, wherein a substrate for the said labeled immunoglobulin antibody is a chemiluminescent or a fluorescent phosphatase, a peroxidase substrate or a fluorescent dye labeled with Digoxigenin (DIG), anti-Digoxigenin, biotin, avidin or streptavidin.

29. The kit of claim 28, wherein said fluorescent phosphatase substrate is ELF 97 phosphatase substrate.

30. The kit of claim 21, wherein the folic acid is labeled with a fluorescent dye, an alkaline phosphatase or a horseradish peroxidase.

31. The kit of claim 30, wherein a substrate for the enzyme labeled folic acid is a fluorescent phosphatase or a chemiluminescent horseradish peroxidase.

32. The kit of claim 21, wherein the folate binding protein in the kit is labeled with biotin and deposited on streptavidin-coated plates.

33. The kit of claim 32, wherein the streptavidin-coated plates in the kit are microtiter plates.

34. The kit of claim 20, wherein said folate binding protein is isolated from vertebrate species.

35. The kit of claim 34, wherein said folate binding protein is isolated from a vertebrate species selected from the group consisting of human, mouse, cow, pig and monkey.
